(19) United States
(12) Patent Application Publication
(10) Pub. No.: US 2023/0245769 A1
(43) Pub. Date: Aug. 3, 2023

(54) DIGITAL HEALTH PLATFORM FOR ARTIFICIAL INTELLIGENCE BASED SEIZURE MANAGEMENT

(71) Applicant: EnlitenAI Inc., Tracy, CA (US)

(72) Inventor: Himanshu Misra, Tracy, CA (US)

(73) Assignee: EnlitenAI Inc., Tracy, CA (US)

(21) Appl. No.: 17/991,038

(22) Filed: Nov. 21, 2022

Related U.S. Application Data
(60) Provisional application No. 63/281,945, filed on Nov. 22, 2021.

(51) Int. Cl. G16H 40/67; A61B 5/00

(52) U.S. Cl. CPC G16H 40/67; A61B 5/0022

(57) ABSTRACT

Implementations described and claimed herein provide systems and methods for a cloud-based seizure management platform for personalized management of seizures while providing assured connectedness across multiple stakeholders. The systems and methods address the needs of a patient in the area of seizure management, through such functionalities as seizure detection, seizure histories and other health-related data management, stakeholder connectedness, tachyphylaxis detection, drug titration guidance, and/or treatment aggressiveness management. The system provides for access for multiple parties, including allowing neurologists and/or caregivers to monitor progression of neurological conditions on a continuous basis while at home or otherwise remote from the patient. The unique methodology of the seizure management system includes wearable technologies coupled with artificial intelligence, machine-learning algorithms, and data modeling techniques to enable personalization of care.

### CROSS-REFERENCE TO RELATED APPLICATION

This application is related to and claims priority under 35 U.S.C. § 119(e) from U.S. Pat. Application No. 63/281,945 filed Nov. 22, 2021 entitled “Al based digital health platform for vitals monitoring using wearable technology for personalization of treatment/care for seizure management”, the entire contents of which is incorporated herein by reference for all purposes.

### FIELD

Aspects of the present disclosure relate generally to systems and methods for a health platform for management of seizures and other medical conditions. More particularly, the present disclosure provides for an artificial intelligence based digital health platform for monitoring of patient vitals via wearable technology for personalization of treatment and/or care for seizure management of the patient.

### BACKGROUND

Strides have been made to bring the diagnosis and management of epilepsy and/or other health-related concerns in line with current technology. However, oftentimes patient diagnostic information is lost or received too late to enact meaningful responses to the patient’s care. For example, one of the biggest challenges faced by patients/caregivers is their inability to share context rich episode specific information with a neurologist right away. Today, the communication typically happens typically 6-8 months after an event at the time of a clinical visit. The patients/caregivers end up relying on their ability to recollect specific details or on their efforts in maintaining diaries/logs. Since the neurologist is rarely present at the time of a seizure as it may occur anytime, their ability to characterize a seizure and build a treatment plan is severely limited. Neurologists find it difficult to monitor the progression of their patients’ neurological condition, especially because clinical observations occur so infrequently. Additionally, each patient responds differently to different classes of anti-seizure medications, therefore, treatment plans cannot be generalized across all patients.

Currently, there are seizure diary applications that are primarily mobile-based apps and allow patients/caregivers to create a journal of the seizure events and logs of specific details. Sensors/wearables have consistently improved their sensor technology and are getting reliable and affordable, but the focus of such devices remains primarily on the detection of a seizure. Currently, many neurologists lack of visibility of episode-specific details and trends relative to the progression of epilepsy. This results in extremely long drug trial-and-error titration cycles that limit the effectiveness of such processes. In addition, the cost burden for insurance carriers associated with epilepsy has skyrocketed for both the carrier and the patient due to the lack of proper seizure management.

It is with these observations in mind, among others, that various aspects of the present disclosure were conceived and developed.

### SUMMARY

Implementations described and claimed herein address the foregoing problems by providing systems and methods for managing health-related data of a patient. One particular implementation may include a system comprising a communication interface receiving biometric data from a wearable device and associated with the patient, one or more data processors, and a non-transitory computer-readable storage medium containing instructions. When the instructions are executed by the one or more data processors, the data processors may detect, based on a comparison of the biometric data to stored baseline vitals data associated with the patient, a seizure event of the patient and transmit, to a computing device, an alert message indicating the detection of the seizure event of the patient. The data processors may also receive, via a user interface in communication the system via the communication interface, additional seizure event data comprising at least one indicator of a potential cause of the seizure event of the patient and correlate and store the biometric data and the additional seizure event data in a database of seizure event data.

In another implementation, a computer-implemented method for seizure management of a patient is provided. The method may include the operations of communicating, via a communication interface, with a wearable biometric device worn by the patient to receive biometric data associated with the patient, inputting the received biometric data to a seizure detection algorithm to compare the biometric data to stored baseline vitals data associated with the patient to detect an occurrence of a seizure event, and transmitting, to a computing device, an alert message indicating the detection of the seizure event of the patient based on an output of a likelihood of a seizure event received from the seizure detection algorithm. The computer-implemented method may also include the operations of receiving, via a user interface executed on the computing device, additional seizure event data comprising at least one indicator of a potential cause of the seizure event of the patient and correlating the biometric data and the additional seizure event data in a database of seizure event data.

Yet another implementation may include One or more tangible non-transitory computer-readable storage media storing computer-executable instructions for performing a computer process on a computing system. The computer process may include the operations of communicating with a wearable biometric device worn by a patient to receive biometric data associated with the patient, inputting the received biometric data to a seizure detection algorithm to compare the biometric data to stored baseline vitals data associated with the patient to detect an occurrence of a seizure event, and transmitting, to a computing device, an alert message indicating the detection of the seizure event of the patient based on an output of a likelihood of a seizure event received from the seizure detection algorithm. The computer process may also include the operations of receiving, via a user interface executed on the computing device, additional seizure event data comprising at least one indicator of a potential cause of the seizure event of the patient, encrypting the correlated biometric data and the additional seizure event data, and storing the biometric data and the additional seizure event data in a database of seizure event data.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

The various features and advantages of the technology of the present disclosure will be apparent from the following description of particular embodiments of those technologies, as illustrated in the accompanying drawings. It should be noted that the drawings are not necessarily to scale; however, the emphasis instead is being placed on illustrating the principles of the technological concepts. The drawings depict only typical embodiments of the present disclosure and, therefore, are not to be considered limiting in scope.

FIG. 1 shows an example network environment that may implement various systems and methods discussed herein.

FIG. 2 shows an example block diagram of a seizure management health platform for managing seizure and other health concerns of a patient.

FIG. 3 shows an example block diagram of a dashboard engine of the seizure management health platform of FIG. 2.

FIG. 4 shows an example block diagram of a seizure tracking engine of the seizure management health platform of FIG. 2.

FIG. 5 shows an example block diagram of an operation of a vitals management engine of the seizure management health platform of FIG. 2.

FIG. 6 shows an example block diagram of an operation of a quality-of-life scoring engine of the seizure management health platform of FIG. 2.

FIG. 7 shows an example block diagram of an operation of a health outcomes scoring engine of the seizure management health platform.

FIG. 8A shows an example block diagram of a drug titration engine of the seizure management health platform of FIG. 2.

FIG. 8B shows illustrates a graph of clinical management of a patient’s seizures based on information and data obtained by the seizure management application.

FIG. 9 shows an example flowchart of a method for a seizure management health platform to collecting and processing patient data for seizure event prediction.

FIG. 10 shows an example flowchart of a method for a teaching an artificial intelligence system for seizure event prediction and others.

FIG. 11 shows an example screenshot of a user interface of a homepage of the seizure management health platform.

FIGS. 12A and 12B show example screenshots of a user interface of the seizure management health platform displaying information associated with a recorded seizure event.

FIGS. 13A-13C show example screenshots of a user interface of the seizure management health platform for managing one or more medications of a patient.

FIG. 14 shows an example screenshot of a user interface of the seizure management health platform displaying seizure event history information.

FIG. 15 shows an example computing system that may implement various systems and methods discussed herein

### DETAILED DESCRIPTION

Aspects of the present disclosure involve systems and methods for a cloud-based seizure management system or platform for personalized management of seizures while providing assured connectedness across multiple stakeholders. Such a system may provide closed loop continuous monitoring of vitals and other biometrics of a patient/user of the system from wearables while simultaneously generating continuous insights using one or more artificial intelligent or machine learning engines. In particular, the seizure management system may correlate vital parameters and associated trends with clinical observations and deviations in specific vitals to build analytical models to detect seizures in a patient and perform propensity analyses. The system may be designed for ambulatory use and at home while allowing for neurologists or other medical professionals to perform continuous assessments of the progression of patient symptoms rather than rely on observations at discrete points of time, such as 6-8 months out.

The seizure management system described herein is aimed at addressing the needs of a patient in the area of seizure management, which may include seizure detection, seizure histories and other health-related data management, stakeholder connectedness, tachyphylaxis detection, drug titration guidance, and/or treatment aggressiveness management. The system provides for access for multiple parties, including allowing neurologists to monitor progression of neurological conditions on a continuous basis while at home or otherwise remote from the patient. The unique methodology of the seizure management system includes wearable technologies coupled with artificial intelligence, machine-learning algorithms, and data modeling techniques to enable personalization of care. The seizure management system also provides for communication of potential distress triggers by patients (non-verbal or speech impaired and/or special needs population) using neurological biomarkers and threshold deviations of specific physiological/vitals to help in propensity assessments for onset of epileptic episodes.

The seizure management system disclosed herein includes a design methodology that allows for seamless data flow and stakeholder specific continuous insight generation that extends patient care beyond seizure detection and seizure diary creation to the broader domain of personalized seizure management. Such personal management may include, but is not limited to, tachyphylaxis onset detection, drug titration guidance, treatment aggressiveness management, and Quality of Life (QoL) balancing.

In one implementation, the seizure management system generates patient specific seizure diaries automatically, including a seizure detection algorithm that detects correlates vitals/biometrics, as well as provides the ability for caregivers or other stakeholders to flag certain events as psychogenic epileptic episodes with non-neurological origin. This methodology allows for seizure detection model enhancements for corrections and/or causation through a machine learning process. Seizure triggers may be specific to a patient and dependent heavily on how each patient responds to certain socio-environmental stimuli such that the seizure detection algorithm may be specifically tailored to a patient or user of the system. Other aspects of the seizure detection algorithm may be based on multiple users of the system.

In some instances, the seizure management system and methodology integrates and aggregates a comprehensive panel of episode specific context rich information from wearables, clinical observations, and electronic medical records/electronic health records (EMR/HER) systems through semantic searches to enable enhanced personalized care by multi-specialty physicians to address multiple co-morbidities associated with epilepsy/seizure management. The methodology of seizure management system facilitates correlational analyses, causations, and propensity analyses using patient specific histories of seizure detections and personalized threshold violations. The threshold violations may be dynamically reviewed by a neurologist or other healthcare provider, who may dynamically change the threshold limits, which help in further personalizing the various prediction models of the system. In general, the seizure management system methodology is designed so that a healthcare provider or other stakeholder receives the right information at the right time to take the right decisions for improving health outcomes on a personalized basis.

As discussed, the seizure management system may include one or more machine learning or artificial intelligence algorithms to aid the system in seizure detection and management. In some implementations, the system may ingest and aggregate feedback data from any combination of wearables or other input sources to enhance the model capabilities. For example, smartwatches and clinical observations from caregivers may be provided to aid in a data building phase. While any additional sensor driven wearable significantly enhances the machine learning models during the model training stage, the absence of these does not reduce the effectiveness of the system. Further, with aggregation of sufficient data and associated learning models, the seizure management system is able to generate prediction models based on propensity analyses involving trends observed in several vitals and other neurological biomarkers.

The seizure management system described herein provides a platform to provide many advantages for stakeholders, including making assessments on the patient’s level of functioning/awareness, building specific customized instruction sets or assessment tests (depending on the patient’s ability to comprehend instructions, allows scoring of a patient’s performance on a relative scale, storing of video/audio logs for each assessment, automatic building of a history of QoL assessments to be used for a drug titration process, establishing treatment aggressiveness limits, and determining the overall Health Outcome Score.

In some instances, the seizure management system may utilize unique data encryption/user authentication capabilities that enable stakeholders to access patient specific episode related information in order to create personalized protocols and treatment plans. The architecture allows for secure data flow and access by specific stakeholders using need-based protocols. The security system may include user authentications, personal identifiable information (PII), patient approval-based access, and/or cybersecurity considerations, etc.

In addition, the seizure management system allows for customizations and creation of comprehensive hypotheses to accommodate specific needs of clinical studies and drug efficacy trials conducted by pharmaceutical and biomedical devices companies. The methodology enables easy integration with genomic databases to study genetic predispositions relevant to certain types of epileptic and links global Neurological databases of the entire patient base to enable Neurologists to leverage this platform for research pursuits.

These and other advantages may become apparent from the discussion included herein.

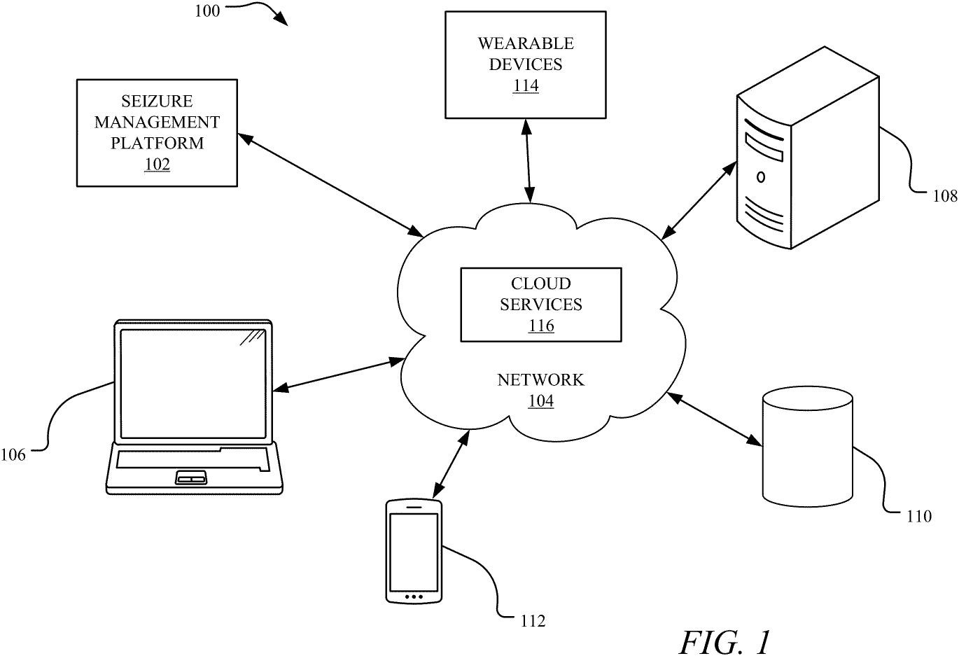

To begin a detailed discussion of an example seizure management system 100, reference is made to FIG. 1. In particular, FIG. 1 illustrates an example network environment for implementing the various systems and methods, as described herein. As depicted, a network 104 is used by one or more computing or data storage devices for implementing the systems and methods for a health management platform, such as a seizure management platform. In one implementation, various components of the seizure management platform 102, one or more user devices 106, one or more databases 110, other network components or computing devices, one or more user mobile devices 112, and/or one or more wearable devices 114 described herein are communicatively connected to the network 104. Examples of the user devices 106 include a terminal, personal computer, a tablet, a mobile computer, a workstation, and/or the like. Examples of mobile devices 112 may include a smart-phone or other type of mobile computing device. Examples of wearable devices 114 may include electronic headbands, smartwatches, wristbands, electronic eyewear, smart clothing, skin patches, or any other electronic devices configured to be worn on the body of a patient.

A server 108 may, in some instances, host the system. In one implementation, the server 108 also hosts a website or an application that users may visit to access the network environment, including the seizure management health platform 102. The server 108 may be one single server, a plurality of servers with each such server being a physical server or a virtual machine, or a collection of both physical servers and virtual machines. In another implementation, a cloud hosts one or more components of the system. For example, cloud services 116 may be offered by the network 104 to host any aspect of the operation and/or component of the mobile health system or seizure management platform 102, such as storage capabilities, compute capabilities, networking capabilities, and the like. Cloud services 116 may host a portion or all of the varied components of the seizure management platform 102 to provide a cloud-based solution. The seizure management platform 102, the user devices 106, the server 108, the mobile devices 112, the wearable devices 114, and other resources connected to the network 104 may access one or more additional servers for access to one or more websites, applications, web services interfaces, etc. that are used for well sequencing and unconventional reservoir management.

The data transferred to and from various devices in operating environment 100 can include secure and sensitive data. Therefore, it can be desirable to protect transmissions of such data using secure network protocols and encryption and to protect the integrity of the data when stored on the various computing devices within the software deployment system. For example, a file-based integration scheme or a service-based integration scheme can be utilized for transmitting data between the various computing devices. Data can be transmitted using various network communication protocols. Secure data transmission protocols and/or encryption can be used in file transfers to protect the integrity of the data, for example, File Transfer Protocol (FTP), Secure File Transfer Protocol (SFTP), and/or Pretty Good Privacy (PGP) encryption. In many implementations, one or more web services can be implemented within the various computing devices. Web services can be accessed by authorized external devices and users to support input, extraction, and manipulation of data between the various computing devices in the operating environment 100. Web services built to support a personalized display system can be cross-domain and/or cross-platform and can be built for enterprise use. Such web services can be developed in accordance with various web service standards, such as the Web Service Interoperability (WS-I) guidelines. Data can be transmitted using the Secure Sockets Layer (SSL) or Transport Layer Security (TLS) protocol to provide secure connections between the computing devices. Web services can be implemented using the WS-Security standard, which provides for secure SOAP messages using XML encryption. In still other examples, a security and integration layer can include specialized hardware for providing secure web services. For example, secure network appliances can include built-in features such as hardware-accelerated SSL and HTTPS, WS-Security, and/or firewalls. Such specialized hardware can be installed and configured in the operating environment 100 in front of one or more computing devices describe herein such that any external devices can communicate directly with the specialized hardware.

It will be appreciated that the network connections shown are illustrative and other means of establishing a communications link between the computers can be used. The existence of any of various network protocols such as TCP/IP, Ethernet, FTP, HTTP and the like, and of various wireless communication technologies such as GSM, CDMA, WiFi, and WiMAX, is presumed, and the various computing devices described herein can be configured to communicate using any of these network protocols or technologies.

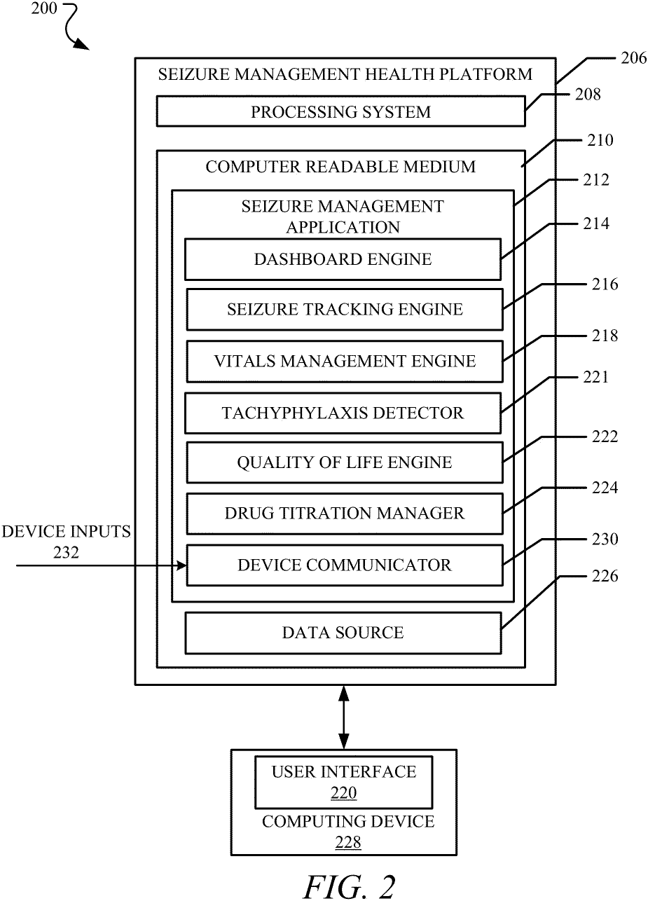

FIG. 2 shows an example block diagram of a seizure management system 200 for managing seizure and other health concerns according to the present disclosure. In general, the system 200 may include a seizure management health platform 206. In one implementation, the seizure management health platform 206 may be a part of the seizure management platform 102 of FIG. 1. As shown in FIG. 2, the seizure management health platform 206 may be in communication with a computing device 228 providing a user interface 220. The computing device 228 may be, in one particular implementation, a mobile device such as a smartphone or a tablet device configured to render the user interface 220. As explained in more detail below, the seizure management health platform 206 may be accessible to various users via the user interface 220 to provide management of seizure treatment and/or any other health related services. In some instances, access to the seizure management health platform 206 may occur through the user interface 220 executed on the computing device 228. Further, the computing device 228 may be operated by any number of users/actors associated with providing healthcare related services, such as a patient, caregiver, physician, therapist, insurance computer or agent, and the like. In this manner, the user interface 220 may be utilized on multiple computing devices 228, including both mobile and non-mobile computing devices, to access the features and services provided by the seizure management health platform 206.

The seizure management health platform 206 may include a seizure management application 212 executed to perform one or more of the operations described herein. The seizure management application 212 may be stored in a computer readable media 210 (e.g., memory) and executed on a processing system 208 of the seizure management health platform 206 or other type of computing system, such as that described below. For example, the seizure management application 212 may include instructions that may be executed in an operating system environment, such as a Microsoft Windows™ operating system, a Linux operating system, or a UNIX operating system environment. By way of example and not limitation, non-transitory computer readable medium 210 comprises computer storage media, such as non-transient storage memory, volatile media, nonvolatile media, removable media, and/or non-removable media implemented in a method or technology for storage of information, such as computer readable instructions, data structures, program modules, or other data.

The seizure management application 212 may also utilize a data source 226 of the computer readable media 210 for storage of data and information associated with the seizure management platform 206. For example and as explained in more detail below, the seizure management application 212 may store received data or inputs, processing details, and/or output information, and the like associated with any aspect of healthcare and/or seizure treatment management. As described in more detail below, data associated with seizure tracking, patient vitals, tachyphylaxis detection, and/or drug titration may be stored and accessed via the user interface 220, among many other types of data. Data stored in the data source 226 may include any type of data associated with the seizure management health platform 206, including vital data, video files, audio files, log information, image files, and the like.

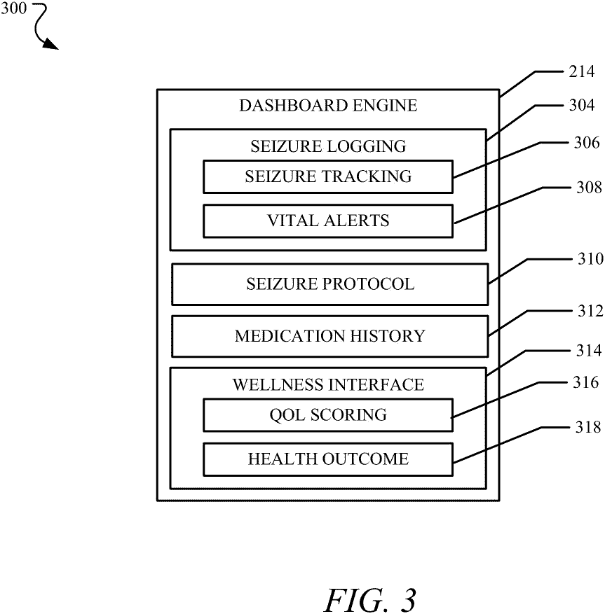

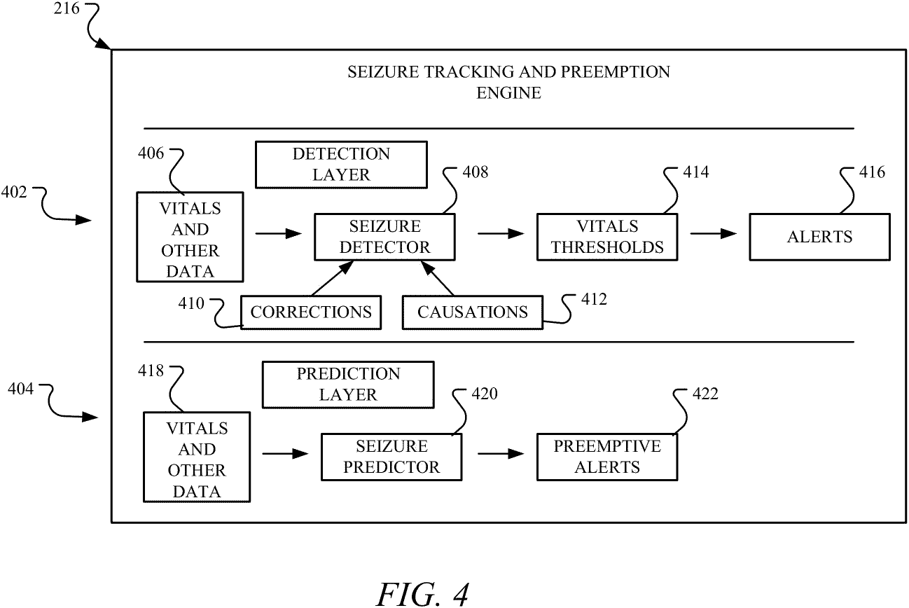

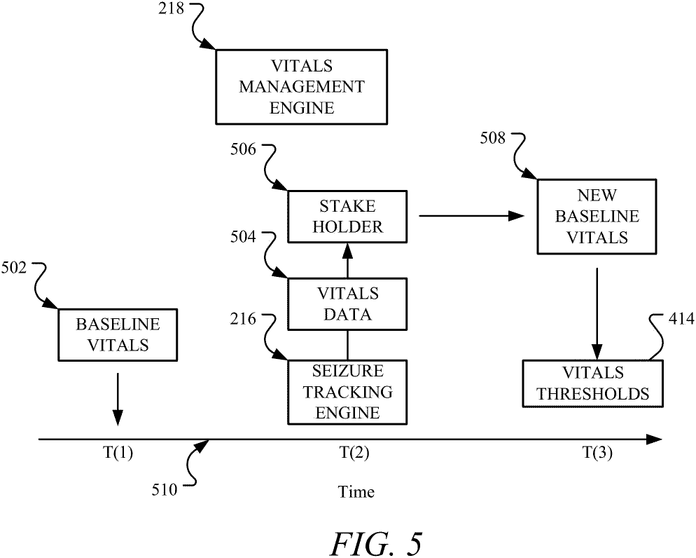

The seizure management system 200 includes methodology and components that allow for seamless data flow and stakeholder specific continuous insight generation that extends patient care beyond detection to the broader domain of personalized seizure management, including tachyphylaxis onset detection, drug titration guidance, treatment aggressiveness management, and Quality of Life (QoL) balancing. To provide these features, the seizure management application 212 may include several components to address the challenges and incorporate such considerations in well sequencing. For example, the seizure management application 212 may include a dashboard engine component 214 for communicating with the user interface 220 to provide access to the seizure management application 212 to a user of the computing device 228 or the computing device itself. For example, FIG. 3 illustrates a block diagram 300 of a dashboard engine 214 of the seizure management health platform 206 of FIG. 2. The illustrated dashboard engine 214 illustrates a few of the many capabilities, components, and/or features of the dashboard engine of the seizure management health platform 206. In addition, many of the components of the dashboard engine 214 may communicate with or otherwise interact with other components and/or features of the seizure management health platform 206. For example, some implementations of the dashboard engine 214 may include a seizure logging component configured to receive information and/or data associated with a seizure event of a patient and store or log the information and/or data with the seizure management health platform 206. In some implementations, the seizure logging 304 may include or communicate with a seizure tracking engine 216 and/or a vitals management engine 218 of the seizure management application 212 to track and log seizure events and activities of the patient. Both of the seizure tracking engine 216 and the vitals management engine 218 are discussed in more detail below with reference to FIGS. 4 and 5, respectively.

In general, the seizure logging 304 feature of the dashboard engine 214 maintains historical data associated with one or more seizure events. For example, the seizure logging 304 may maintain historical data for all seizure types, including specific types of seizures, date and time of occurrence, audio files, video files, log files, vital readings (such as electroencephalogram (EEG) data, heartbeat data, blood pressure, temperature, blood oxygen levels, etc.), vital threshold information received at the vital alerts 308 component, possible seizure triggers, signs of distress in the patient, and the like. As discussed in more detail below, such information may be provided to the seizure logging 304 through a mobile device, a wearable device, or any other type of computing device. The seizure logging 304 may also provide access to such logged data for any stakeholder of the seizure management health platform 206, such as a caregiver, the patient, a neurologist or other health professional, insurance personnel, and the like. In some implementations, one or more types of seizure data may be restricted such that access to the data is limited to authorized stakeholders. In general, any person or party provided access to the seizure management health platform 206 may or may not be provided access to seizure data through the seizure logging 304 of the dashboard engine 214.

The dashboard engine 214 may further include a seizure protocol 310 component that provides one or more protocols to be conducted or performed in response to a seizure event. In some instances, the protocol may be based on a determined severity of the seizure event. For example, the seizure protocol 310 may provide instructions, via the user interface 220, to log or enter specific information into the seizure logging component 304 for a mild seizure event. For more severe seizures, the seizure protocol 310 may provide instructions to administer emergency medication, contact the patient’s physician, contact emergency personnel, take the patient to the emergency room, and the like. In general, the seizure protocol 310 may provide instructions for responding to any type of seizure event and may be specific to a particular patient or may be based on data collected for multiple patients. In one implementation, the provided instructions may be based on a machine learning technique and may be altered over time based on historical seizure events for a particular patient or collection of patients, previous instructions provided, and information associated with an outcome of the seizure events. For example, the seizure protocol 310 may provide care instructions for a determined low-risk seizure and receive, perhaps from feedback through the user interface 220 or through one or more wearable devices, the patient’s vitals after the seizure event. Based on the feedback data, the seizure protocol 310 may adjust the provided instructions for responding to a future low-risk seizure event. Such adjustment may include changes to a type and amount of medication prescribed in response to the event and/or changes to an identity of a medical personnel to contact. For example, the instructions provided in one instance may suggest immediately contacting emergency personnel. However, based on feedback post event, the instructions may be adjusted to suggest contacting the patient’s neurologist at the earliest possible convenient time. Such feedback information may be related the patient’s seizure event or related to seizure events over a subset of all users of the seizure management health platform 206. Other aspects of the seizure management health platform 206 may also be adjusted through one or more machine learning techniques, as described in more detail herein.

The dashboard engine 214 may also include a medication history 312 portal through which a stakeholder may access data and/or information of medications prescribed to the patient. In one implementation, the medication history 312 portal may communicate with a medication adherence engine, discussed in more detail below. In general, the medication history 312 portal may provide access to a patient’s current list of medications, history of prescribed medications, history of medication changes, history of missed dosages, and the like. As above, such information may or may not be available to any stakeholder associated with the patient. Some stakeholders may be restricted from accessing such information based on an authentication procedure executed by the dashboard engine 214. Additional information associated with the medication history 312 and medication adherence engine are described in more detail below.

In addition, the dashboard engine 214 may include a wellness interface 314 for accessing data and/or information associated with a quality of life or level of wellness for the patient. For example, the wellness interface 314 may interact or communicate with a quality-of-life scoring engine 316 to obtain a quality-of-life (QOL) score for the patient. In general, the QOL score indicates a quality of a patient’s life while being treated for seizures and/or other health-related conditions. The QOL score may be calculated as discussed in more detail below and compared to a threshold value corresponding to a desired QOL for the patient. The QOL score may be aspiration and include more than management of the patient’s seizures. Rather, the QOL may comprise additional considerations, such as the patient’s happiness, the patient’s freedom to move about freely, the patient’s level of comfort at any moment or over a period of time, indicators of the patient’s improvement, and the like. Each of these indicators and more may be considered in the QOL score, which may be presented to a stakeholder via the wellness interface 314 managed by the dashboard engine 214. The wellness interface 314 may also display a health outcome 318 associated with a patient’s seizure health, including management through medication, time between seizure events, and the like. The health outcome 318 may be displayed in the user interface 220 of the computing device 228. Additional wellness information may also be displayed and/or managed by the dashboard engine 214, including individualized wellness and/or QOL goals and any other information useful to a stakeholder using the seizure management health platform 206.

Returning to the seizure management system 200 of FIG. 2 and as discussed above with relation to the dashboard engine 214, the seizure management application 212 may include a seizure tracking engine 216 to track occurrences of seizure events and/or predict the occurrence of a seizure event. FIG. 4 illustrates one example of a seizure tracking engine 216 of the seizure management health platform 206 of FIG. 2. The seizure tracking and preemption engine 216 of FIG. 4 includes both a detection layer 402 for detecting a seizure event for a patient and a prediction layer 404 for predicting the occurrence of a future seizure event. The seizure tracking engine 216 may include more or fewer functionalities than discussed herein with reference to FIG. 4.

The detection layer 402 of the seizure tracking engine 216 may detect, based on information and data received at the seizure management application 212, the occurrence of a seizure event of a patient associated with the seizure management health platform 206. For example and as described in more detail below, a patient may register with the seizure management application 212 through the user interface 220 to track seizure events. Once registered, information and data potentially associated with a seizure event may be provided to the seizure tracking engine 216. In one particular implementation, vitals and other data 406 may be provided to the seizure tracking engine 216. Such information may be collected and/or tracked through one or more wearable devices attached to the patient, such as a smartwatch, electronic headband, wristband, electronic eyewear, smart clothing, etc. Upon collecting, the wearable devices may transmit the data 406 to the seizure management health platform 206 to reach the seizure tracking engine 216 through any wired or wireless connection between the wearable devices and the seizure management health platform. Other data may also be provided, such as inputs provided the seizure management health platform 206 via the user interface 220 by the caregiver, the patient, a third party, or any other method for providing information to the seizure management health platform.

The seizure tracking engine 216 may process the received vitals and other data 406 to determine if a seizure event is occurring. In one implementation, a seizure detector 408 may compare the vitals and other data 406 to one or more threshold values to detect the occurrence of a seizure. For example, a patient’s EEG activity data 406 may be obtained from a wearable device and provided to the seizure tracking engine 216. The seizure detector 408 may compare the received EEG activity data 406 to a threshold EEG value and, if the received data meets or exceeds the threshold value, a seizure event may be occurring. Additional vital and other data 406 may also be compared to corresponding threshold values by the seizure detector 408. In this manner, the seizure detector 408 may be an algorithm or model that receives as inputs the patient vitals and other data 406 and outputs a likelihood of a seizure event occurring. In one implementation, the seizure detection algorithm 408 may include a machine learning technique for analyzing historical data to develop an algorithm for seizure detection. For example, the seizure detector 408 may compare feedback information, such as an indication that a seizure event occurred, to the determination by the algorithm that a seizure was occurring. A positive correlation indicates that the algorithm is accurate in determining the occurrence of a seizure while a negative correlation indicates that the threshold values of the algorithm may need to be adjusted to improve the accuracy of the detector. In some implementations, the feedback information used to train the seizure detector 408 may include corrections 410 to the algorithm received via the user interface 220 from a user of the computing device 228. For example, the corrections 410 may include an indicator that a seizure event occurred and a severity of the seizure event. Other corrections 410, such as a false reading, malfunctioning wearable device, the occurrence of a seizure event when one was not detected, and the like may also be provided. In some instances, the corrections 410 may be received from the wearable devices or other computing devices. Other feedback information may also be provided. For example, one or more causations 412 may be provided to the seizure detector 408 algorithm indicating one or more conditions that caused or may cause a seizure event, such as certain stimuli experienced by the patient just prior to a seizure event. With this and other feedback information, the seizure detector 408 may be adjusted to improve the accuracy of the seizure detection.

The seizure detector 408 may output one or more threshold values that, taken together, may indicate the occurrence of a seizure event. These output values may be compared to one or more vital thresholds 414. In circumstances in which the output values from the seizure detector 408 meet or exceed the vitals threshold values 414, a seizure event may be detected as occurring. Alternatively, if the output values of the seizure detector 408 do not exceed the vitals threshold values 414, a seizure event may not be occurring. In instances in which a seizure event is detected, one or more alerts 416 may be generated by the seizure management health platform 206. The alerts 416 may take many forms. For example, an alert may be displayed in the user interface 220 of the computing device 228 in response to the comparison of the output of the seizure detector 408 to the vitals threshold values 414. In another example, a communication, such as a text alert, email, phone call, etc., may be conducted to one or more stakeholders of the health platform 206, such as a caregiver of the patient, a physician, emergency personnel, and the like. In general, the seizure tracking engine 216 may generate any electronic communication to alert a party to the occurrence of a seizure by the patient.

In addition to a detection layer 402, the seizure tracking engine 216 may include a prediction layer 404 configured to predict the occurrence of a seizure before the event happens. In the example illustrated in FIG. 4, the prediction layer 404 may receive the same or similar vitals and other data 418 as the detection layer 402, including but not limited to data from one or more wearables and other inputs to the seizure management health platform 206. The vital and other data 418 may be provided to a seizure predictor 420 algorithm. The seizure predictor 420 may operate in a similar manner as the seizure detector 408 described above, except the algorithm may predict the likelihood of a seizure event based on the vitals and other data 418. Also similar to above, the seizure predictor 420 may be a machine learning algorithm that receives feedback, such as an accuracy of a seizure event prediction, and adjusts one or more parameters of the algorithm based on the feedback. For example, the seizure predictor 420 may output a high likelihood of a seizure event based on the vitals and other data 418. However, a seizure event may not occur and feedback indicating the missed prediction may be provided to the predictor 420, such as through the data obtained from the wearable devices and/or inputs through the user interface 220. The seizure predictor 420 may then be adjusted in response to the feedback information to improve the accuracy of the predictor. In some instances, accuracy feedback of predictors for other patients may also be used to adjust the seizure predictor 420.

Based on the output of the seizure predictor 420, one or more preemptive alerts 422 may be generated. For example, in circumstances in which the seizure predictor 420 indicates a likely seizure, one or more preemptive alerts 422 may be generated. As above, such alerts may include a communication, such as a text alert, email, phone call, etc., to one or more stakeholders of the health platform 206, such as a caregiver of the patient, a physician, emergency personnel, and the like. In general, any alert may be generated by the seizure tracking engine 216 in response to an output from the seizure predictor 420.

In some instances, the seizure tracking engine 216 may utilize a vitals management engine 218 of the seizure management application 212. FIG. 5 illustrates an example block diagram of an operation of a vitals management engine 218 of the seizure management health platform 206 of FIG. 2. In particular, the block diagram of FIG. 5 illustrates the vitals management engine 218 updating one or more vitals threshold values based on vitals data provided to the seizure management application 212 through a wearable device and/or through the user interface 220. The vitals management engine 218 may provide additional functionalities and features to users of the seizure management health platform 206, as discussed herein.

The operations of the vitals management engine 218 are illustrated along a timeline 510 of arbitrary length. At a first time T(1), a patient or other user may register with the seizure management health platform 206 and one or more baseline vital values 502 may be provided or obtained. For example, the user may associate a wearable device with the seizure management health platform 206 and one or more vital data may be provided by the wearable device to the platform. In another example, baseline vital information or data 502 may be input to the seizure management health platform 206 via the user interface 220 executed on the computing device 228, such as by a patient, caregiver, physician, etc. At some later time T(2), the seizure tracking engine 216 may detect a seizure event and vitals data 504 may be provided to the vitals management engine 218. The vitals data 504 may comprise any data associated with the vitals of a patient, such as vitals information at the time of the detected seizure, trends and averages over a period of time prior to and post the seizure event, summaries of vitals information, raw vitals data over a period of time associated with the detected seizure activity, and the like. One or more stakeholders 506 may access such vitals information and data. For example, a neurologist may utilize the seizure management health platform 206 to access the vitals information and data associated with the seizure event. In another implementation, a computing device or algorithm associated with the seizure management health platform 216 or vitals management engine 218 may receive the vitals data 504 associated with the seizure tracking engine 216.

Through an analysis of the vitals data 504, one or more new baseline vital values 508 may be set corresponding to the vitals associated with the seizure activity. For example, the vitals information may indicate patient conditions that correspond to a seizure event such that the baseline, or “normal”, vital data for the patient may be adjusted for the patient. The new baseline vitals 508 may indicate a resting condition for the patient. The vitals data 504, contrarily, indicate conditions in which the patient is experiencing a seizure event. Based on this analysis, the baseline vitals for the patient may be adjusted or a new set of vital data 508 may be determined. Further, at T(3), the new baseline vital values 508 may be utilized to set vital threshold values 414 for use in generating seizure event alerts and other functions of the seizure tracking engine 216 discussed above. For example, the new baseline vitals 508 may be used to adjust the vitals threshold values that indicate a seizure event. In this manner, the vitals management engine 218 may aid the seizure management health platform 206 in detecting and alerting for seizure events.

Returning to the seizure management health platform 206 of FIG. 2, the seizure management application 212 may also include a tachyphylaxis detector 221. Tachyphylaxis is an acute, sudden decrease in response to a drug after its administration, akin to a rapid drug tolerance. The tachyphylaxis detector 221 may invoke seizure episode specific information from the seizure tracking engine 216 as well as the information around the severity of vital parameter violations beyond permissible thresholds to arrive at data driven estimations on when tachyphylaxis may set in. For example, the tachyphylaxis detector 221 may analyze such data as the severity, intensity, type, and duration of a seizure event, in addition to the number of seizure events occurring within a time period, such as per week. The tachyphylaxis detector 221 may also analyze other data, such as co-morbidity effects, vitals information, prescribed drugs, dosages, frequency of drug administration, and the like. With such data, the tachyphylaxis detector 221 may predict or otherwise output a likelihood of an onset of tachyphylaxis for the patient. As such, the tachyphylaxis detector 221 may be an algorithm that inputs various data tracked or received by the seizure management health platform 206 and outputs a likelihood or other indicator of tachyphylaxis of a patient. In general, the tachyphylaxis detector 221 may receive any information obtained by the seizure management application 212 to determine the onset of tachyphylaxis. Further, the tachyphylaxis detector 221 may be a machine learning algorithm that receives feedback on the accuracy of a prediction of tachyphylaxis occurring and adjusts one or more parameters of the algorithm based on the feedback. Such feedback information may be associated with the specific patient for which a tachyphylaxis detection is occurring or from any number of users associated with the seizure management health platform 206. In this manner, the tachyphylaxis detector 221 may be an analytical and statistical model to evaluate characteristics of seizure events and provide a prediction and/or detection of tachyphylaxis in a patient.

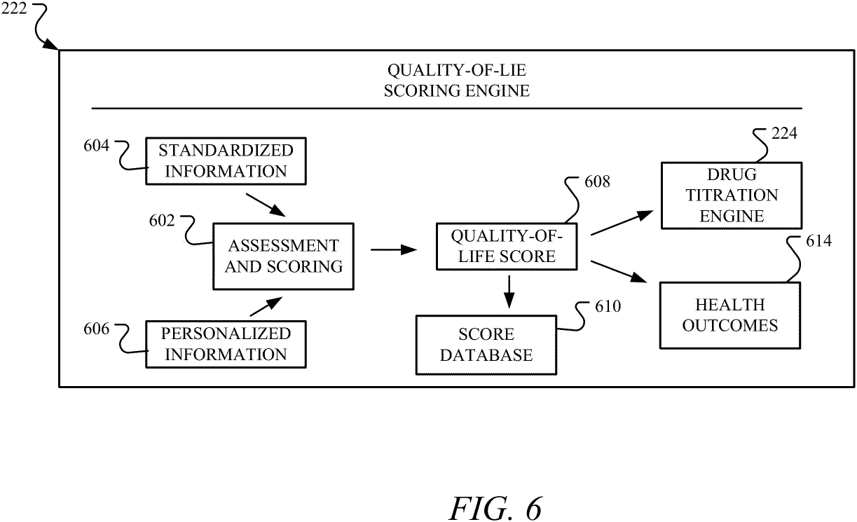

FIG. 6 shows an example block diagram of operations of a quality-of-life (QoL) scoring engine 222 of the seizure management application 212 of FIG. 2. The QoL scoring engine 222 may aid a user of the seizure management health platform 206 to determine a level of awareness/functioning of a patient, despite the fact that the patient may be exhibiting a reduction of the incidence of seizures. Owing to the side effects of many of the seizure control medications, many patients may experience extreme fatigue/drowsiness that may prevent them from performing basic functions, including impaired swallow functioning. These could result in aspiration leading to pneumonia which may, in turn, trigger more seizures. Thus, although medications may be useful in controlling seizures in a patient, the patient’s overall QoL may be reduced and alternative treatments may be explored or determined for the patient to improve the QoL score for the patient. In other instances, the QoL scoring engine 222 may be beneficial in guiding drug titration exercise, as prescribing physicians may continually assess how aggressive to be in attempts to manage seizures while simultaneously maintaining a particular QoL for the patient.

In one implementation, the QoL scoring engine 222 may include an assessment and scoring component 602 configured to generate a QoL score 608 for a patient based on input information and/or data. For example, a standardized test may be presented to a patient or other stakeholder associated with the seizure management health platform 206 to obtain standardized information 604 or feedback data on a QoL of the patient. In one implementation, the standardized test 604 may be displayed in the user interface 220 of the seizure management application 212 and answers or other inputs to the test may be entered via an input device of the computing device 228. In addition, personalized information 606 of a patient that may be beyond the standardized information may also be provided to the assessment and scoring component 602. In some instances, a caregiver may invoke standardized tests (as per Occupational therapist recommendations/supervision) or, better still, create their own set of personalized tests. Many times, patients/children respond better to familiar tests that their caregivers/family have constructed knowing the potential of each patient. The tests may be specific assessments developed by a caregiver to assess performance which can be built into each patient’s dashboard as a customization to gather personalized information 606 of the patient. In some instances, a history of each of these assessments may be recorded as a series of audio and/or video recordings that may be viewed and compared at any time.

The assessment and scoring component 602 may process the standardized information 604 and/or the personalized information 606 to output a QoL score 608. In general, the assessment and scoring component 602 may comprise an algorithm that inputs the standardized information 604 and/or the personalized information 606 and outputs a QoL score 608 for a particular patient. The QoL score 608 may be any value, such as a numerical value on a 1-100 scale, although other methods for indicating a general QoL of the patient may be used. Further, the assessment and scoring component 602 may be a machine learning algorithm that receives feedback on the accuracy of the QoL score 608 and adjusts one or more parameters of the algorithm based on the feedback. Such feedback information may be associated with the specific patient’s QoL or from any number of users associated with the seizure management health platform 206. In this manner, the assessment and scoring component 602 may be an analytical and statistical model to evaluate received information and/or data and provide a QoL score 608 associated with a patient. In some instances, the QoL score 608 may be stored in a score database 610 and made accessible to users of the seizure management health platform 206. One or more historical QoL scores 608 may be made available to the users of the seizure management health platform 206 for comparison.

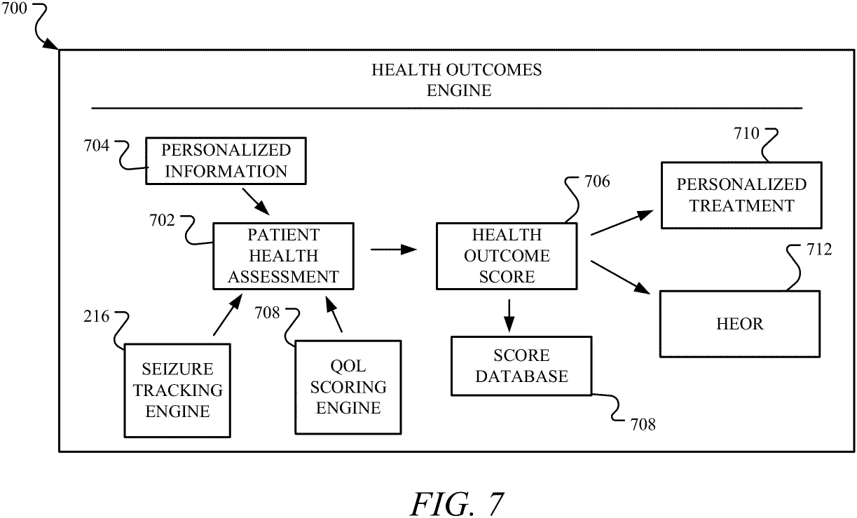

In addition to calculating and storing the QoL score 608, the QoL scoring engine 222 may also provide the score to one or more other engines of the seizure management application 212. For example, the QoL scoring engine 222 may provide the score 608 to a drug titration engine 224 and/or a health outcomes engine 614. FIG. 7 illustrates shows an example block diagram of an operation of such a health outcomes scoring engine 700 of the seizure management health platform 206. Similar to above, the health outcomes engine 700 may aid a user of the seizure management health platform 206 to assess the overall wellbeing of a patient in a holistic sense. In general, the health outcomes score may factor in seizure history (such as seizure count, intensity, durations etc.), the QoL score 608 determined by the QoL scoring engine 222, and any other information or data obtained by the seizure management health platform 206.

As illustrated in FIG. 7, the health outcomes engine 700 may include a patient health assessment component 702 to generate a health outcome score 706 for a patient. In one implementation, the patient health assessment 702 may receive personalized information 704 of a patient. Such personalized information 704 may include, for example, personalized goals and individual assessments of the patient’s health. Personalized goals may include indicators of particular QoL score, one or more responses to a questionnaire by the patient or other stakeholders interacting with the user interface 220 of the seizure management application 212, or any other information that may be personalized to a particular patient. In addition, the patient health assessment 702 may receive information or data from the seizure tracking engine 216, such as seizure count, intensity, durations, seizure detections and corresponding vitals information, etc. The patient health assessment 702 may also receive a QoL score 608 from the QoL scoring engine 222, as described above. The patient health assessment 702 may utilize these inputs to generate a health outcome score 706 associated with the patient. The health outcome score 706 may be any value, such as a numerical value on a 1-100 scale, although other methods for indicating a health outcome of the patient may be used. Further, the patient health assessment 702 may be a machine learning algorithm that receives feedback on the accuracy of the health outcomes score 706 and adjusts one or more parameters of the algorithm based on the feedback. Such feedback information may be associated with the specific patient’s health outcome score or from any number of users associated with the seizure management health platform 206. In this manner, the patient health assessment 702 may be an analytical and statistical model to evaluate received information and/or data and provide a health outcome score 706 associated with a patient. Further, the health outcome score 706 may be stored in a health score database 708 and made accessible to users of the seizure management health platform 206. One or more historical health outcomes scores 706 may be made available to the users of the seizure management health platform 206 for comparison.

In addition to calculating and storing the health outcomes score 706, the health outcomes engine 700 may also provide the health outcomes score to one or more other components of the seizure management health platform 206. For example, the health outcomes score 706 may be processed into a personalized treatment 710 procedure for the particular patient. In one implementation, a personalized seizure management and treatment procedure may be established for the patient, such as by a physician or other caretaker of the patient. The health outcome score 706, providing a more holistic view of the patient’s treatment, may therefore be considered when generating the patient personalized treatment 710 In another example, the health outcomes score 706 may be provided to one or more insurance companies or other third parties for health economics and outcomes research (HEOR) to aid in generating understanding of the effects of a new drug or intervention for the treatment of seizures. In general, the health outcome score 706 may be made available to any stakeholder or user of the seizure management health platform 206.

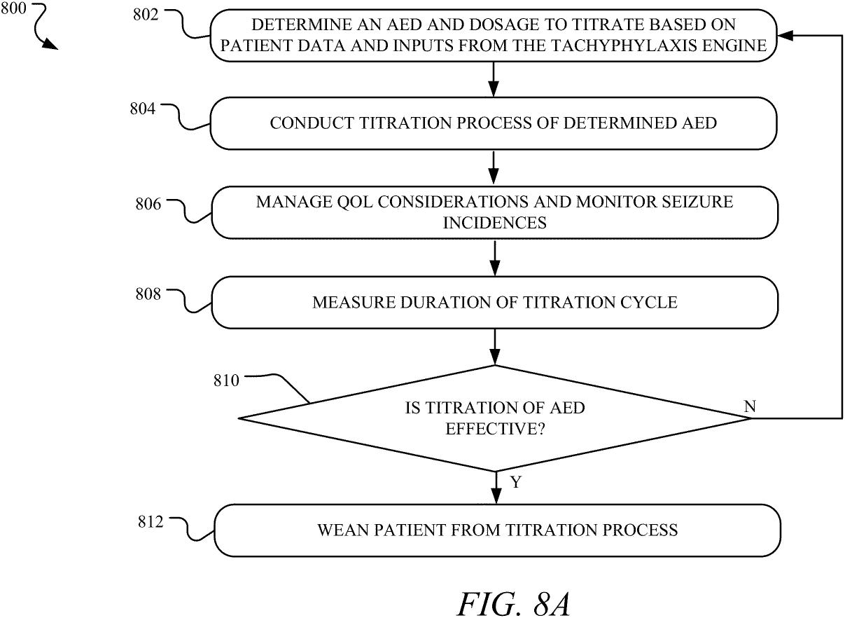

Returning to the seizure management health platform 206 of FIG. 2, the seizure management application 212 may also include a drug titration manager 224 to monitor and detect potential drug titration opportunities of medications, such as anti-epileptic drugs (AEDs) prescribed to the patient. In general, drug titration is the process of adjusting the dose of a medication dose to achieve the best response in the patient while minimizing adverse effects. As such, the drug titration manager 224 may obtain and process medication information for a patient or patients and identify optimal titration opportunities. In particular, FIG. 8A illustrates a flowchart of a method 800 for an operation of a drug titration engine 224 of the seizure management application 212. The operations of the method 800 of FIG. 8A may be performed by the seizure management application 212 and, in particular, by the processing system 208 executing instructions stored in the computer readable medium 210. More or fewer operations may be included in the method 800 and executed by the seizure management application 212 and/or the drug titration manager 224.

Beginning at operation 802, an AED may be determined for a titration process based on patient data and inputs from other engines or components of the seizure management application 212, such as the tachyphylaxis detector 221. The tachyphylaxis detector 221 may aid in identifying an AED for titration, particularly an AED that may be losing effectiveness. In one particular example, FIG. 8B illustrates a graph 820 of clinical management of a patient’s seizure activity based on information and data obtained by the seizure management application 212. In particular, the graph 820 illustrates an AED titration cycle in response to patient data received at the seizure management application 212. In the implementation illustrated, the graph 820 includes a first portion 822 illustrating seizure data of a patient received at the seizure tracking engine 216 of the application 212. Although any patient information and data discussed above may be considered, the graph portion 822 plots a patient’s blood oxygen levels (indicated by the green line 840) over a 52-week period. Other time periods may also be considered by the seizure management application 212. The blood oxygen levels for the patient may be received, in some instances, through a wearable device associated with the patient that provides blood oxygen level data to the seizure management application 212, as discussed above. The graph portion 822 may also include data received through the user interface 220. Other data, such as the classification of seizure events by the seizure tracking engine 216, may also be displayed. In the example illustrated, a number of seizure events indicated as “severe” by the seizure tracking engine 216 are graphed as line 842. Other data and information from the seizure tracking engine 216 or any other component of the seizure management health platform 206 may be considered by the drug titration manager 224.

A second portion 824 of the graph 820 illustrates a drug treatment for the patient. In particular, a series of bar graphs illustrate the amount of several drugs prescribed to the patient over the 52-week period. An amount prescribed for each of drug 826-834 is illustrated as different shades of gray within each bar of the bar graph 824, although different colors may also be used. In general, a larger portion of each bar of a particular shade corresponds to a higher dosage of that drug prescribed to the patient. Although five AEDs are illustrated in the graph portion 824, it should be appreciated that any number of drugs at any type of dosage may be considered by the drug titration engine 224 of the seizure management application 212 to identify an AED for titration or for other drug interaction purposes. It should also be appreciated that the seizure management application 212 may not generate the graph or make visual the patient-related data. Rather, the graph 820 is provided for illustrative purposes to aid in explaining the method 800 of FIG. 8A.

Returning to operation 802 of the method 800, the drug titration engine 224 may analyze patient data, such as the seizure data illustrated in portion 822 of graph 820, and/or data from the tachyphylaxis detector 221 to determine an AED for titration. For example, the tachyphylaxis detector 221 and/or the drug titration engine 224 may identify an increase in seizure events for a patient, such as at time 844 illustrated in portion 822 in which the patient’s blood oxygen level lowers significantly and there is an increase in the number of severe seizure events. The tachyphylaxis detector 221 may determine that a tachyphylaxis of one or more of the patient’s drugs may have occurred. Further, the tachyphylaxis detector 221, the drug titration engine 224, or a stakeholder associated with the patient (such as the patient’s caregiver, neurologist, physician, etc.) may identify one an AED drug of the plurality of drugs 826-834 prescribed to the patient for titration. In response and at operation 804, a titration process of the determined AED may be conducted at time 846. In general, the titration process may include an adjustment to a dosage of one or more of the drugs 826-834 prescribed to the patient and may be determined by any qualified party or system of the seizure management health platform 206, such as a neurologist associated with the patient.

At operation 806, the drug titration engine 224 may monitor aspects of a patient’s seizure health during the titration period. For example, the drug titration engine 224 may manage a QoL score generated by the QoL engine 222 and/or monitor seizure activity based on information received from the seizure tracking engine 216. In addition, at operation 808, the drug titration engine 224 may monitor or measure the duration of the titration process of the determined AED. At operation 810, the drug titration engine 224 may determine if the titration process is effective for reducing the seizure activity of the patient. If the patient’s seizure activities continue at an above-normal rate, the titration process may be adjusted by returning to operation 802 where a new dosage of the determined AED may be prescribed or a new AED may be identified and adjusted. This trial and error period for the titration process may continue for a period of time 836, with adjustments to the type and amount of drugs provided to the patient and a monitoring of the effects of the drugs on the patient’s seizure events. The identification of the AED for titration and the changes in doses may again be performed by any qualified party or system associated with the seizure management health platform 206.

At some later time (such as time 848), it may be determined that the drug titration process is complete as the patient’s data returns to a normal state. In such circumstances, the drug titration engine 224 or a qualified stakeholder may begin a process of weaning the patient off of the titrated AED. The period following the titration period 836 is illustrated in graph 820 as time 848 during which the dosages of the prescribed drugs 826-834 may return to pre-titration levels or be adjusted according to any prescription for the patient. In this manner, the drug titration engine 224 may provide an interface through which one or more drug titration periods may be conducted for a patient or other user of the seizure management health platform 206.

In some instances, the patient’s data and/or information may indicate another round of drug titration as determined by the drug titration engine 224. For example, another increase in determined severe seizure events (such as at time 850) may determine a new period 838 of drug titration occur. The drug titration engine 224 may perform the same method 800 as described above to adjust the drug dosages and types given to the patient in response to the drug titration period 838. In some instances, the drug titration process may lead to alternative AEDs provided to the patient. For example, a QoL score for the patient may indicate that the side effects for a particular AED is uncomfortable for the patient. In response, the titration process may include removing a particular AED or prescribing another AED or other drug to address the QoL score and/or seizure events. In general, any changes to the cocktail of drugs prescribed to the patient may be conducted based on the drug titration engine 224.

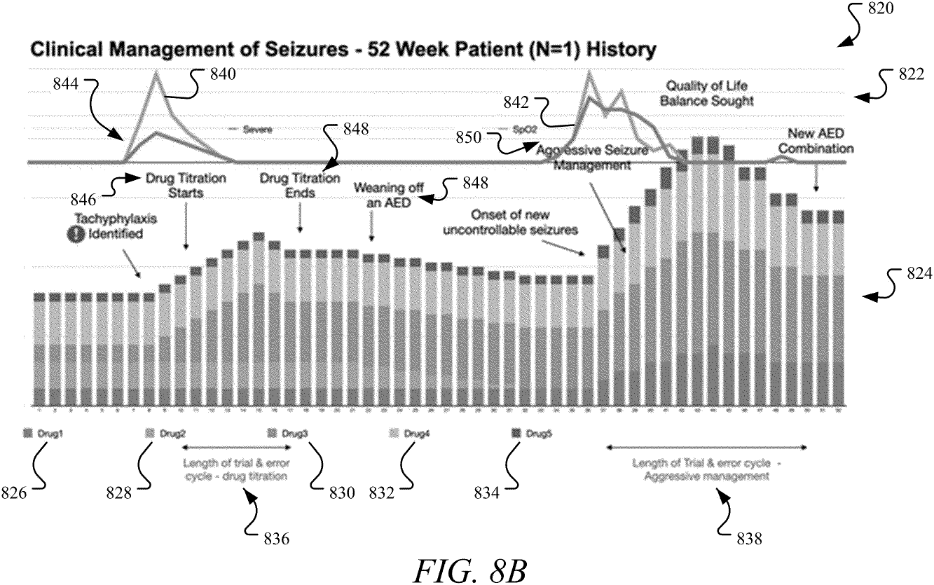

Turning to FIG. 9, a flowchart of a method 900 for a seizure management health platform to collecting and processing patient data for seizure event prediction is disclosed. The operations of the method 900 may be performed by any component or components of the seizure management health platform 206. In one particular implementation, one or more operations may be performed by the seizure tracking engine 216 of the seizure management application 212, although other implementations are contemplated.

Beginning in operation 902, medical history data or other relevant data of a particular patient may be received at the seizure management health platform 206. For example, medical records of a patient may be received from any electronic medical records (EMR) and/or electronic health records (EHR) source or computing system. In one implementation, the EMR/EHR system may provide the medical history data or other data via the user interface 220 or computing device 228 in communication with the seizure management health platform 206. Thus, such medical history data may be provided automatically through a communication between the seizure management health platform 206 and an EMR/EHR system. In other instances, a user of the computing device 228 or the seizure management health platform 206 may provide the medical history data.

At operation 904, vitals data and/or other biometrics obtained from one or more wearable devices and associated with the patient may be provided to the seizure management health platform 206. Such wearable devices may include, but are not limited to, smartwatches, headbands, electronic eyewear, smart clothing, and the like. For example, the seizure management application 212 may include a device communicator 230 for communicating with one or more external devices or systems. Such external devices may include a wearable device, such as a headband. The wearable device may be registered with the device communicator 230 or the seizure management health platform 206 through a registration process executed on the computing device 228 and via the user interface 220. Once registered, the wearable device may begin providing vitals data 232 to the device communicator 230 of the seizure management application 212. Any number of such devices may be registered with the seizure management application 212 to provide vitals data 232 of the patient to the seizure management application. Such vitals data may be obtained in real-time for monitoring of a condition of the patient.

At operation 906, additional patient-related data and/or files may be received at the seizure management health platform 206. For example, a stakeholder associated with the patient, such as a caregiver, may provide context rich information pertaining to the patient’s condition. Such data may include audio files, video files, images, log files, and the like that provide information on the condition of the patient. In general, the seizure management health platform 206 may receive and store any type of information and/or data or files to provide information and understanding to a patient’s condition.

At operation 908, the received patient data and information may be processed for seizure detection and other uses. For example, and as described above, such information may be analyzed by the seizure tracking engine 216 to detect the occurrence of a seizure event. In addition, the patient data and/or information may be available to certain users of the seizure management health platform 206. For example, the caregiver data may be streamed or otherwise made available to a patient’s neurologist for analysis and diagnostics of the patient. In other instances, the data may be categorized and/or stored for use in generating historical data of the patient’s journey. In general, the data and information may be utilized by the seizure management health platform 206 for any purposes described herein and others

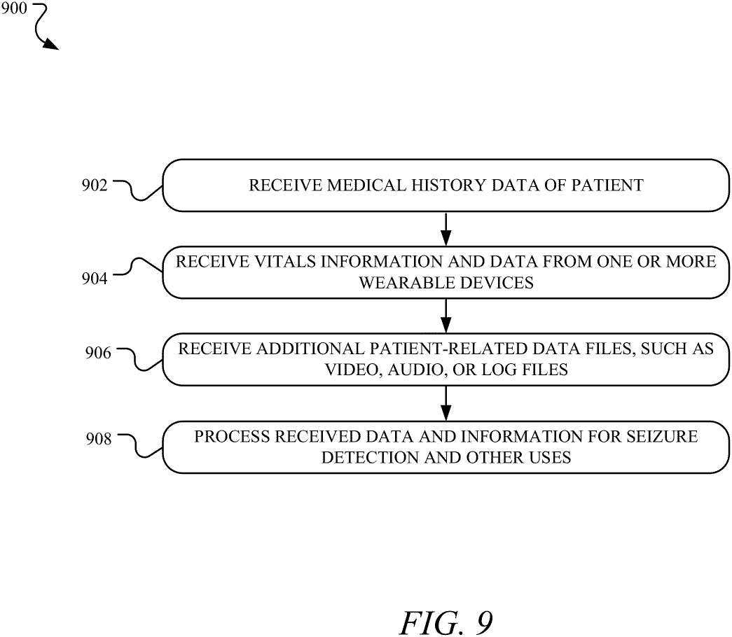

FIG. 10 is an example flowchart of a method 1000 for a teaching an artificial intelligence system for seizure event prediction and components of the seizure management health platform 206. The method 1000 may be utilized by the seizure management health platform 206 to update any algorithm utilized by the seizure management application 212 to improve the accuracy of the algorithm. Thus, although described herein as improving the seizure tracking engine 216 or algorithm, the method 1000 may be applied to any such algorithm.

Beginning in operation 1002, an initial prediction model may be generated, such as a model to determine the occurrence of a seizure event or predict the occurrence of a seizure event. The model may receive, as inputs, data associated with the predicted event, such as patient vitals or other health-related information, and output some value, such as the likelihood that a seizure event is occurring or will occur in the near future. In many instances, the initial prediction model may take the form a machine-learning model or an artificial intelligence machine including one or more parameters or operations that are adjustable based on feedback information associated with an accuracy of the provided output from the model. At operation 1004, the model may output an initial prediction based on the provided inputs, such as a likelihood of an event occurring or the detection of an event.

At operation 1006, feedback associated with an accuracy of the initial provided output may be received. Such feedback may include an indication of a correct prediction, a false positive, a false negative, a percentage associated with an accuracy measurement, and the like. In general, any feedback associated with the output of the initial prediction output may be received. At operation 1008, one or more parameters, operations, conditions, steps, etc. of the prediction model may be adjusted based on the received feedback. The adjustment to the prediction model may be made to improve the accuracy of the prediction model. For example, a false positive feedback indication may result in the adjustment of a parameter of the model to prevent the false positive from re-occurring. The feedback may also indicate one or more inputs that are more dispositive to an accurate prediction and the adjustment to the model may include associating a higher weighted value to those inputs for future predictions. Regardless, the model may be adjusted in some manner in response to the received feedback data and/or information. In many instances, the operations of the method 1000 of FIG. 10 may be repeated to finetune the prediction model for a more accurate prediction of potential events.

The communications transmitted to and from the seizure management health platform 102 may be secure transmissions between components and data stored in any of the devices involved in the operation of the seizure management health platform may be encrypted to provide secure operation platform. In one implementation, data transferred between the seizure management health platform and the cloud services 116 or any other communication with the network 104 may be double encrypted using an asymmetric and symmetric encryption at both ends of the communication. For asymmetric encryption, a private key and a public key may be generated on the seizure management health platform 102 and the cloud services 116. The public key may be shared between each device for decryption while the private key of the seizure management health platform and public key of the cloud services 116 may be stored in the keychain, which is a privacy database in the seizure management health platform that cannot be breached. In one particular implementation, the encryption follows a 2048-bit Secure Sockets Layer (SSL) protocol. For symmetric encryption, a symmetric key may be used to encrypt the data that is being sent to the cloud services 116 from the seizure management health platform 102 and vice versa. In one implementation, the symmetric key may be a random 30 character length key generated at every request for communications. Data from the seizure management health platform 102 to the cloud service 116 may be first encrypted with the asymmetric key and then the symmetric key before transfer, and transmitting through a HyperText Transfer Protocol Secure (HTTPS) Application Programming Interface (API) call for added security of the transferred data. Encryption of data stored at the seizure management health platform 102 and the cloud services 116 is also provided.

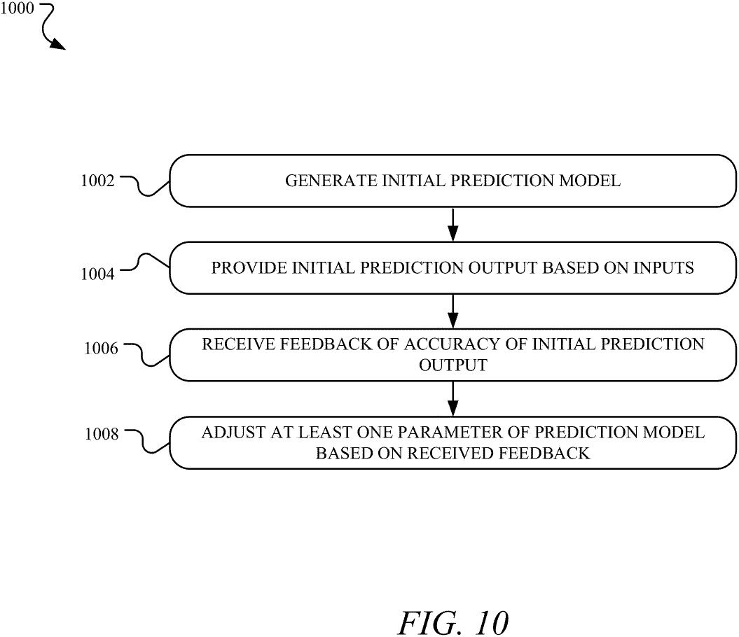

FIG. 11 shows an example screenshot of a user interface 1100 of a homepage of the seizure management health platform. The user interface 1100 may be displayed on a display device of the computing device 228 in communication with the seizure management health platform 206 or any other computing device associated with the health platform. Further, the user interface 1100 illustrated is but one example of a homepage of the seizure management health platform 206 and more or fewer items may be displayed in the user interface. In the example shown, several components of the user interface 1100 may provide information to a user of the seizure management health platform 206, such as a patient, caregiver, physician, etc. For example, the user interface 1100 may include a calendar portion 1102 displaying one or more dates, including today’s date. A user may horizontally scroll through the calendar portion 1102 to past dates to access seizure-related information associated with those past dates. In this manner, a history of a patient’s activities may be accessible through the user interface 1100.

The user interface 1100 may also include summary of seizure information 1104 for the date selected in the calendar portion 1102. In the example shown, the seizure summary information 1104 may display a number of reported or detected seizures, a number of reported or detected severe seizure events, a number of reported or detected emergency calls made, and/or a number of reported or detected hospitalizations, all for the selected date in the calendar portion 1102. A third portion 1106 may provide a link to access past history of seizures.

As mentioned above, a stakeholder or user of the seizure management health platform 206 may provide information associated with seizure activities of a patient. In some instances, such information may be provided through the user interface 1100. For example, the user interface 1100 may include an activation button 1108 through which a user of the seizure management health platform 206 may add new seizure event information. Selection of the button 1108 may transition the user to a new user interface, discussed in more detail below. The user interface 1100 may also display a video capture link 1110 to quickly capture a video of the patient during a seizure event. Selection of the video capture link 1110 may launch a video application on the computing device 228 through which a video of the patient’s seizure event may be recorded. The inclusion of the video capture link 1110 on the homepage provides for quick selection to ensure enough video information of the event may be captured. A navigation bar 1112 may provide quick access to multiple functionalities of the seizure management health platform 206, such as the homepage 1100 of FIG. 11.

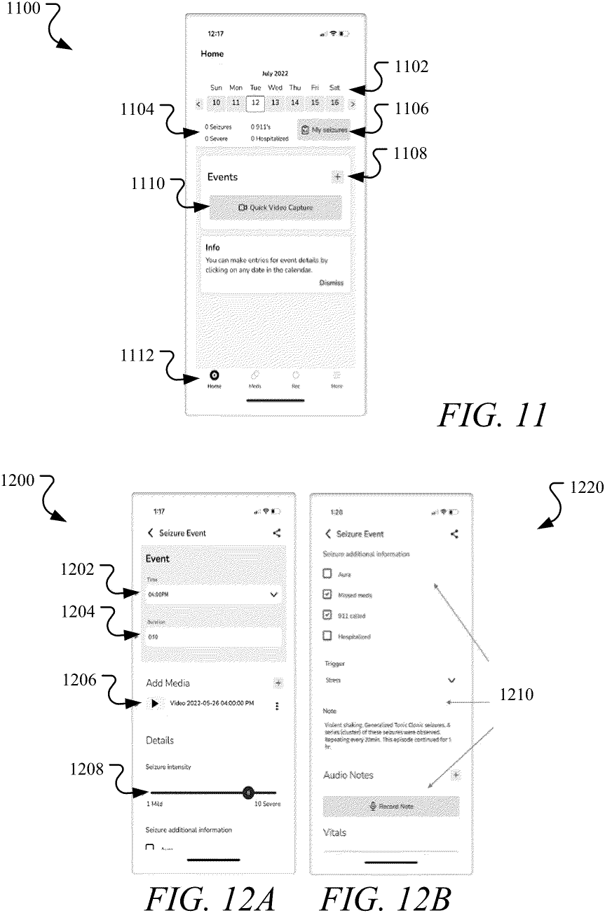

FIGS. 12A and 12B show example screenshots of a user interface of the seizure management health platform displaying information associated with a recorded seizure event. In one implementation, the screenshots 1200, 1220 may be accessed through the activation button 1108 discussed above. Upon selection of the activation button 1108, screenshot 1200 may be displayed. The screenshot 1200 may provide several areas in which seizure event information may be provided. For example, a time when a seizure is detected may be entered in box 1202 and/or a duration of the seizure may be entered in box 1204. In addition, any audio or video files of the event may be uploaded through the add media portion 1110 of the screenshot. Such files may include any digital file associated with capturing aspects of the seizure event, such as an audio or video recording of the event. Further, an intensity of the seizure event may be entered through seizure selector 1208 of the user interface 1200. Scrolling down on the user interface may display interface 1220 through which more seizure event information may be entered. For example, one or more determined triggers and/or notes of observations by a caregiver or other witness, either through text or audio file, may be provided through screenshot 1220. In general, the user interface may include any number of portions to receive additional information associated with a seizure event.

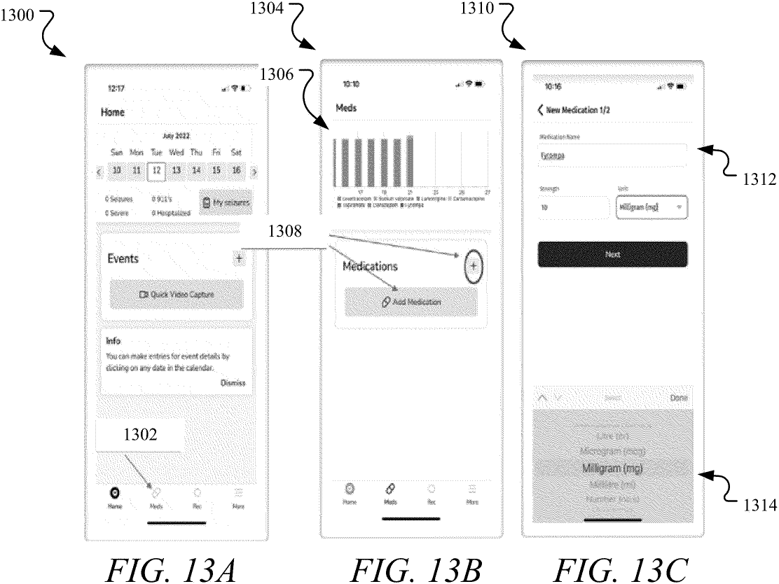

FIGS. 13A-13C show example screenshots of a user interface of the seizure management health platform for managing one or more medications of a patient. To access the medication management from the homepage (illustrated as screenshot 1300), a user may select the medication tab 1302 at the bottom of the homepage 1300. Upon selection, medicine management screenshot 1304 may be displayed. Through the medicine management screen 1304, a user may manage the medications for a particular patient. The currently prescribed medications may be displayed in a first portion 1306, perhaps in a bar graph with different medications illustrated as different colors. Additional medications may be added by selecting the add medication buttons 1308. Selection of either of the add medication buttons 1308 may launch the add medication screen 1310, which may include a portion 1312 for identification of a prescribed medication and a portion 1314 for providing a portion size. In general, the user interface may be used to manage a patient’s prescribed or taken medications as part of the management of the patient’s health.

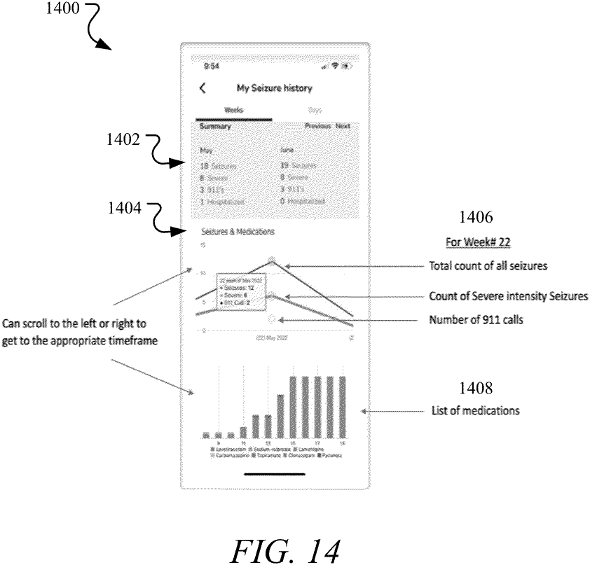

FIG. 14 shows an example screenshot of a user interface of the seizure management health platform displaying seizure event history information. Through the user interface 1400, a user of the seizure management health platform 206 can receive a summary of seizure history for a patient. In particular, the screenshot 1400 may include a first portion 1402 that summarizes seizure events by weeks, days, or months. Another portion 1404 may include scrollable graphs that illustrate the patient’s seizure history. For example, a line graph 1406 may be presented that includes several features, such as a total number of all seizures, a count of severe seizures, a number of emergency calls, etc. for a given past week. A bar graph of the medications 1408 taken by the patient during the week may also be displayed. The user of the seizure management health platform 206 may scroll left or right in the second portion 1404 to access seizure history information for other weeks to gain a broad understanding of the patient’s activities and the history of the patient’s condition over time. As above, more or less information may be presented in the screenshot 1400 of FIG. 14 to show the patient’s seizure history.

Through the systems and methods described above, a cloud-based seizure management system or platform for personalized management of seizures while providing assured connectedness across multiple stakeholders is provided, including closed loop continuous monitoring of vitals and other biometrics of a patient/user of the system from wearables while simultaneously generating continuous insights using one or more artificial intelligent or machine learning engines. The systems and methods are designed to address the needs of a patient in the area of seizure management, through such functionalities as seizure detection, seizure histories and other health-related data management, stakeholder connectedness, tachyphylaxis detection, drug titration guidance, and/or treatment aggressiveness management. The system provides for access for multiple parties, including allowing neurologists and/or caregivers to monitor progression of neurological conditions on a continuous basis while at home or otherwise remote from the patient. The unique methodology of the seizure management system includes wearable technologies coupled with artificial intelligence, machine-learning algorithms, and data modeling techniques to enable personalization of care.

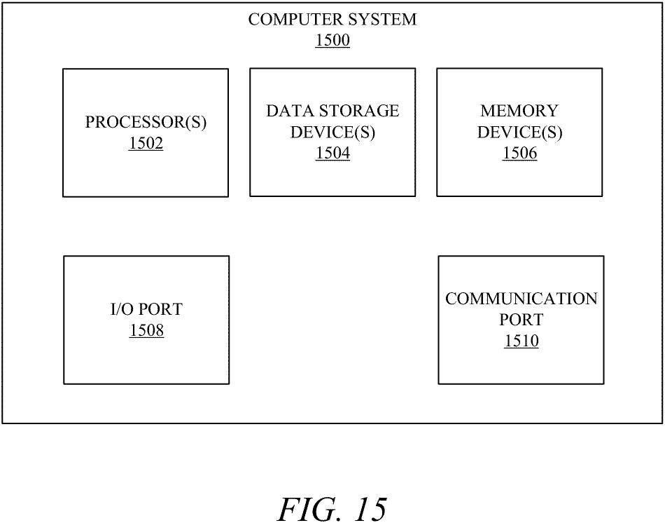

Referring to FIG. 15, a detailed description of an example computing system 1500 having one or more computing units that may implement various systems and methods discussed herein is provided. The computing system 1500 may be applicable to the seizure management platform system 102 of FIG. 1, the system 100, and other computing or network devices. It will be appreciated that specific implementations of these devices may be of differing possible specific computing architectures not all of which are specifically discussed herein but will be understood by those of ordinary skill in the art.

The computer system 1500 may be a computing system is capable of executing a computer program product to execute a computer process. Data and program files may be input to the computer system 1500, which reads the files and executes the programs therein. Some of the elements of the computer system 1500 are shown in FIG. 15, including one or more hardware processors 1502, one or more data storage devices 1504, one or more memory devices 1506, and/or one or more ports 1508-1510. Additionally, other elements that will be recognized by those skilled in the art may be included in the computing system 1500 but are not explicitly depicted in FIG. 15 or discussed further herein. Various elements of the computer system 1500 may communicate with one another by way of one or more communication buses, point-to-point communication paths, or other communication means not explicitly depicted in FIG. 15.

The processor 1502 may include, for example, a central processing unit (CPU), a microprocessor, a microcontroller, a digital signal processor (DSP), and/or one or more internal levels of cache. There may be one or more processors 1502, such that the processor 1502 comprises a single central-processing unit, or a plurality of processing units capable of executing instructions and performing operations in parallel with each other, commonly referred to as a parallel processing environment.

The computer system 1500 may be a conventional computer, a distributed computer, or any other type of computer, such as one or more external computers made available via a cloud computing architecture. The presently described technology is optionally implemented in software stored on the data stored device(s) 1504, stored on the memory device(s) 1506, and/or communicated via one or more of the ports 1508-1510, thereby transforming the computer system 1500 in FIG. 15 to a special purpose machine for implementing the operations described herein. Examples of the computer system 1500 include personal computers, terminals, workstations, mobile phones, tablets, laptops, personal computers, multimedia consoles, gaming consoles, set top boxes, and the like.

The one or more data storage devices 1504 may include any non-volatile data storage device capable of storing data generated or employed within the computing system 1500, such as computer executable instructions for performing a computer process, which may include instructions of both application programs and an operating system (OS) that manages the various components of the computing system 1500. The data storage devices 1504 may include, without limitation, magnetic disk drives, optical disk drives, solid state drives (SSDs), flash drives, and the like. The data storage devices 1504 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1506 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 1504 and/or the memory devices 1506, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 1500 includes one or more ports, such as an input/output (I/O) port 1508 and a communication port 1510, for communicating with other computing, network, or reservoir development devices. It will be appreciated that the ports 1508-1510 may be combined or separate and that more or fewer ports may be included in the computer system 1500.

The I/O port 1508 may be connected to an I/O device, or other device, by which information is input to or output from the computing system 1500. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 1500 via the I/O port 1508. Similarly, the output devices may convert electrical signals received from computing system 1500 via the I/O port 1508 into signals that may be sensed as output by a human, such as sound, light, and/or touch. The input device may be an alphanumeric input device, including alphanumeric and other keys for communicating information and/or command selections to the processor 1502 via the I/O port 1508. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 1500 via the I/O port 1508. For example, an electrical signal generated within the computing system 1500 may be converted to another type of signal, and/or vice-versa. In one implementation, the environment transducer devices sense characteristics or aspects of an environment local to or remote from the computing device 1500, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 1500, such as, physical movement of some object (e.g., a mechanical actuator), heating or cooling of a substance, adding a chemical substance, and/or the like.

In one implementation, a communication port 1510 is connected to a network by way of which the computer system 1500 may receive network data useful in executing the methods and systems set out herein as well as transmitting information and network configuration changes determined thereby. Stated differently, the communication port 1510 connects the computer system 1500 to one or more communication interface devices configured to transmit and/or receive information between the computing system 1500 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 1510 to communicate one or more other machines, either directly over a point-to-point communication path, over a wide area network (WAN) (e.g., the Internet), over a local area network (LAN), over a cellular (e.g., third generation (3G) or fourth generation (4G) or fifth generation (5G) network), or over another communication means. Further, the communication port 1510 may communicate with an antenna or other link for electromagnetic signal transmission and/or reception.

The system set forth in FIG. 15 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A system for managing health-related data of a patient, the system comprising:
a communication interface receiving biometric data from a wearable device and associated with the patient;
one or more data processors; and
a non-transitory computer-readable storage medium containing instructions which, when executed by the one or more data processors, cause the one or more data processors to:
detect, based on a comparison of the biometric data to stored baseline vitals data associated with the patient, a seizure event of the patient;
transmit, to a computing device, an alert message indicating the detection of the seizure event of the patient;
receive, via a user interface in communication the system via the communication interface, additional seizure event data comprising at least one indicator of a potential cause of the seizure event of the patient; and
correlate and store the biometric data and the additional seizure event data in a database of seizure event data.

2. The system of claim 1 wherein the additional seizure event data comprises at least one of a log data file, an audio file, or a video file.

3. The system of claim 1, the instructions further causing the one or more data processors to:
provide the biometric data to a seizure detection algorithm; and
receive, from the seizure detection algorithm, an output indicating a likelihood of an occurrence of the seizure event of the patient.

4. The system of claim 3 wherein the additional seizure event data comprises feedback data of an indication of an accuracy of the detection of the seizure event of the patient.

5. The system of claim 4, the instructions further causing the one or more data processors to:
adjust, based on the feedback data, at least one parameter of the seizure detection algorithm.

6. The system of claim 1, the instructions further causing the one or more data processors to:
classify, based on the comparison of the biometric data to the stored baseline vitals data associated with the patient, a severity of the seizure event of the patient, wherein the alert message indicates the classification of the severity of the seizure event of the patient.

7. The system of claim 1, the instructions further causing the one or more data processors to:
update, based on the received biometric data associated with the patient, the stored baseline vitals data to generate new baseline vitals data for the patient.

8. The system of claim 1 wherein detecting the seizure event of the patient comprises predicting an occurrence of a future seizure event of the patient.

9. The system of claim 1, the instructions further causing the one or more data processors to:
receive and store, in the database of seizure event data, medication data associated with the patient; and
generate a display on the user interface of a visual representation of the medication data associated with the patient.

10. The system of claim 1, the instructions further causing the one or more data processors to:
determine, based on the detection of the seizure event of the patient, a medication associated with the patient for a drug titration process;
monitor the biometric data of the patient over a period of time; and
determine an end of the drug titration process based on the monitored biometric data of the patient.

11. The system of claim 10 wherein determining the end of the drug titration process is further based on receiving, via the communication interface, an indication of a quality-of-life measurement of the patient.

12. The system of claim 10 wherein determining the medication associated with the patient for a drug titration process is further based on determining, based on the comparison of the biometric data to stored baseline vitals data associated with the patient, an onset of tachyphylaxis of the medication for the patient.

13. A computer-implemented method for seizure management of a patient, the computer-implemented method comprising:
communicating, via a communication interface, with a wearable biometric device worn by the patient to receive biometric data associated with the patient;
inputting the received biometric data to a seizure detection algorithm to compare the biometric data to stored baseline vitals data associated with the patient to detect an occurrence of a seizure event;
transmitting, to a computing device, an alert message indicating the detection of the seizure event of the patient based on an output of a likelihood of a seizure event received from the seizure detection algorithm;
receiving, via a user interface executed on the computing device, additional seizure event data comprising at least one indicator of a potential cause of the seizure event of the patient; and
correlating the biometric data and the additional seizure event data in a database of seizure event data.

14. The computer-implemented method of claim 13 wherein the wearable biometric device is one of an electronic headband, a smartwatch, a smart wristband, an electronic eyewear device, a smart clothing device, or an electronic skin patch.

15. The computer-implemented method of claim 13 further comprising:
encrypting, via one or more encryption keys, the alert message transmitted to the computing device; and
encrypting, prior to storing in the database of the seizure event data, the correlated biometric data and the additional seizure event data.

16. The computer-implemented method of claim 13 further comprising:
receiving feedback data of an indication of an accuracy of the detection of the seizure event of the patient; and
altering, based on the feedback data, at least one parameter of the seizure detection algorithm.

17. The computer-implemented method of claim 13 further comprising:
determining, based on the detection of the seizure event of the patient, a medication associated with the patient for a drug titration process;
monitoring the biometric data of the patient over a period of time; and
determining an end of the drug titration process based on the monitored biometric data of the patient.

18. The computer-implemented method of claim 13 further comprising:
receiving via the user interface executed on the computing device, standardized healthcare information associated with the patient and personalized healthcare information associated with the patient; and
generating a quality-of-life score for the patient based on the standardized healthcare information associated with the patient and personalized healthcare information associated with the patient.

19. One or more tangible non-transitory computer-readable storage media storing computer-executable instructions for performing a computer process on a computing system, the computer process comprising:
communicating with a wearable biometric device worn by a patient to receive biometric data associated with the patient;
inputting the received biometric data to a seizure detection algorithm to compare the biometric data to stored baseline vitals data associated with the patient to detect an occurrence of a seizure event;
transmitting, to a computing device, an alert message indicating the detection of the seizure event of the patient based on an output of a likelihood of a seizure event received from the seizure detection algorithm;
receiving, via a user interface executed on the computing device, additional seizure event data comprising at least one indicator of a potential cause of the seizure event of the patient;
encrypting the correlated biometric data and the additional seizure event data; and
storing the biometric data and the additional seizure event data in a database of seizure event data.

20. The one or more tangible non-transitory computer-readable storage media storing computer-executable instructions for performing a computer process on a computing system of claim 19, the computer process further comprising:
authenticating a user with the computing system; and
displaying, on a display device in communication with the computing system, the biometric data and the additional seizure event data of the patient.
